(19) United States
(12) Patent Application Publication
(10) Pub. No.: US 2008/0114266 A1
(43) Pub. Date: May 15, 2008

(54) Inner-Body Sound Monitor and Storage

(76) Inventors: Albert Shen, Atherton, CA (US); Pen C. Li, San Jose, CA (US)

Correspondence Address: NORTH WEBER & BAUGH LLP, 2479 E. BAYSHORE ROAD, SUITE 707, PALO ALTO, CA, 94303, US

(21) Appl. No.: 11/553,926

(22) Filed: Oct. 27, 2006

(51) Int. Cl. A61B 7/00

(52) U.S. Cl. 600/586

(57) ABSTRACT

A system, apparatus and method are described for recording, processing, measuring and transmitting electrical data relating to audible signals originating from within a human body. Acoustic signals originating from a human body are detected by a hand-held device that converts these acoustic signals into corresponding electrical signals. Within the housing of this hand-held device, these signals are processed in the electrical domain, such as filtering, amplifying, storing and analyzing, so that they may be provided in a preferred output format. In certain embodiments of the invention, a speaker on the device housing outputs audible signals relating to the detected inner-body acoustic signals. In other embodiments, a port is provided that outputs electrical signals which may be transmitted over a connection to a distant remote device or stored on a computing device external to the hand-held device. In yet other embodiments, an antenna is provided that transmits a wireless signal relating to the detected inner-body acoustic signals. These various embodiments of the invention allow for a more effective diagnosis of medical conditions, such as asthma or heart ailments, by a remote medical professional or recording of inner-body acoustic signals which may be later analyzed by the medical professional.

### BACKGROUND

A. Technical Field

The present invention relates generally to monitoring of sound originating from a human body, and more particularly, to a simple patient-usable device that records, stores and transmits electrical signals related to acoustic signals originating from inside a body.

B. Background of the Invention

The rising cost of medical care has become an increasing concern in today's society. Health maintenance organizations, medical groups, and insurance companies are attempting to create efficiencies within their medical care practices to maintain high-quality medical care while reducing the underlying costs. Central in the discussion of cost savings is the appropriate and timely utilization of medical services. Lack of appropriate preventive services, delays in diagnosis and treatment, and limited on-going chronic disease management all contribute to the hastening of diseases such as asthma, congestive heart failures, and the likes. It's commonly recognized the majority of the health care expenditure, and a higher level of morbidity and mortality, exist when illness is treated later in the disease process.

Disease management by doctors, nurses and medical support personnel often involve the use of devices, kits or markers to be utilized in both clinical and home settings. For now, prevention, treatment and monitoring of a chronic disease such as asthma involve mostly the use of a patient's own account of cough or breathing difficulties. Along with timely visits to clinicians, recent guidelines from the National Institute of Health calls for the use of peak flow meters or lung-function tests to help clinicians to provide evidence-based remedies in accordance with the level of lung dysfunction. The disparity of this approach is that a majority of children newly diagnosed with the disorder are unable to voluntarily exhale to provide an accurate measurement with any current devices. Also, most patients do not present themselves with symptoms alarming enough to warrant immediate intervention. In the case of asthma, this is especially evident because the majority of these patients have their first symptoms before 5 years of age

Treatment for asthma involves ongoing monitoring, timely intervention and prevention, along with the management of environmental triggers and precipitants. Such as treatment is a daunting task, even for the medical professionals, as it requires a consistent and temporal working interplay between the patient, his/her symptoms record as seen by the care provider and the clinician. After the diagnosis is made, the patients are instructed to use medication based on their severity of symptoms. In times of sickness, especially for younger children, the care providers are left on their own to make that decision without the benefit of a more objective marker for initiating or stopping the treatment. Currently, care providers provide a historical record of triggers or cough-associated symptoms as their guide for treatment, leaving the clinicians to guess at when and what to treat. Late at night, such a decision is even harder to make by parents and providers alike. While many clinicians offer remote advise, much of the time relying on the subjective and inexact observation of a wary and anxious care provider. This reliance tends to lead to inappropriate utilization of the emergency service and contributing to a larger share of the health care expenditure. The number of asthma patient has been increasing, as illustrated in FIG. 1 showing a study of the number of asthmatic individuals done by the National Institute of Health. It is expected that asthma will become even more prevalent within society causing further stress on the current healthcare infrastructure.

Doctors typically use a stethoscope to measure or listen to breathing and lung noises within a patient in order to diagnose asthma. The use of a stethoscope provides a doctor real-time information about a patient's asthmatic condition. Unless properly trained, a parent or caregiver is generally unable to properly use a stethoscope to record or diagnose asthmatic symptoms of an individual. In particular, such a parent or caregiver may be unable to recognize what he/she is hearing within the stethoscope or describe the sounds to a medical profession on the phone.

Because a medical professional is unable to remotely observe or listen to breathing symptoms of a patient, a well-founded diagnosis of an asthma attack is difficult to provide over the phone. As a result, patients (and in particular children) are often asked to come into an emergency room or doctor's office when such a visit is entirely unnecessary. Furthermore, when the patient arrives and asked to describe the asthma attack, he/she may have difficulty in remembering or describing certain details of the attack. As a result, a doctor is often not provided complete information about the attack.

As previously described, the number of asthmatic individuals is rapidly increasing within the population. Providing these individuals medical care does and will continue to place a significant burden on the world's medical systems and infrastructures.

What is needed is systems, apparatuses and methods that address the above-described issues.

### SUMMARY OF THE INVENTION

The present invention provides a system, apparatus and method for recording, processing, measuring and transmitting electrical signals relating to sound originating within a human body. The system, apparatus and method are designed to supplement the diagnoses and treatment of patients by medical professionals by providing these professional recorded inner-body sounds.

In accordance with various embodiments of the invention, the apparatus is a single-housing device that is designed for use by a non-medical individual. The apparatus may be self-administered or used on another individual to record inner-body sounds, such as those associated with breathing and heart symptoms. The apparatus may be a purely analog device or be a digital device. The inner-body sounds may be recorded on a storage device within the apparatus housing in accordance with various techniques and compression methods.

The recorded inner-body sounds may be supplemented with associated data such as the date and time of the recording, the body location from which the recording was taken, patient comments inputted within the apparatus, sound analysis and other types of data. This data may be valuable to the interpretation and analysis of the recorded inner-body sounds by a medical professional.

The recorded inner-body sounds may be provided to a medical professional or replayed to a patient using various methods. In certain embodiments of the invention, the apparatus comprises a speaker that can audibly replay the recorded sounds. In other embodiments, the apparatus comprises an electrical port(s) that facilitates the transmission of an electrical signal of the recorded sounds to a device remote to the apparatus. The port(s) may interface with headphones, networking systems, telephone devices, or other communications media on which an electrical signal may be transmitted. In yet other embodiments, the apparatus comprises an antenna which transmits a wireless signal of the recorded sounds to a device remote to the apparatus. These outputs allow both real-time playback of recorded inner-body sounds and playback at a later time, both of which may be done at the location of the apparatus or at a remote location.

A system including both an inner-body sound monitor and recording apparatus and a remote receiving device allows a medical professional to receive additional information to supplement a diagnosis of a patient. For example, a doctor or nurse, located remote to a patient, is able to listen to inner-body sounds to aid a diagnosis of the patient. Various techniques, some of which are described below, are available to transmit these recorded inner-body sounds to the medical professional.

The system may also be used to supplement a patient's medical record by maintaining historical recordings of a patient's inner-body sounds. For example, breathing recordings or heart recordings may be maintained in order to provide a historical recordation of a patient's particular symptom or symptoms.

### BRIEF DESCRIPTION OF THE DRAWINGS

Reference will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. (“FIG.”) 1 is a chart illustrating statistical information relating to asthma occurrences within the U.S. population.

FIG. 2A is a first exemplary illustration of an inner-body acoustic signal monitor and measuring apparatus according to various embodiments of the present invention.

FIG. 2B is a second exemplary illustration of an inner-body acoustic signal monitor and measuring apparatus according to various embodiments of the present invention.

FIG. 2C is a third exemplary illustration of an inner-body acoustic signal monitor and measuring apparatus according to various embodiments of the present invention.

FIG. 2D is a fourth exemplary illustration of an inner-body acoustic signal monitor and measuring apparatus according to various embodiments of the present invention.

FIG. 2E is a fifth exemplary illustration of an inner-body acoustic signal monitor and measuring apparatus according to various embodiments of the present invention.

FIG. 3A illustrates measurement locations on a human chest from which inner-body acoustic signals may be recorded, stored, and measured according to various embodiments of the invention.

FIG. 3B illustrates measurement locations on a human back from which inner-body acoustic signals may be recorded, stored, and measured according to various embodiments of the invention.

FIG. 4 is a functional block diagram of a basic digital inner-body acoustic signal monitor and measuring apparatus according to various embodiments of the invention.

FIG. 5 is a functional block diagram of a dynamic digital inner-body acoustic signal monitor and measuring apparatus, including an analysis module, according to various embodiments of the invention.

FIG. 6 is a functional block diagram of a basic analog inner-body acoustic signal monitor and measuring apparatus according to various embodiments of the invention.

FIG. 7 illustrates exemplary system applications in which acoustic signals originating from a human body may be transmitted to a remote location according to various embodiments of the invention.

FIG. 8 is a flowchart illustrating a general method for receiving acoustic signals from a human body, processing the signals within the electrical domain and subsequently transmitting an output signal related to the acoustic signals according to various embodiments of the invention.

FIG. 9 is a flowchart illustrating a method for providing recorded inner-body sounds that may be used during a remote diagnosis of an asthmatic patient according to various embodiments of the invention.

FIG. 10 is a flowchart illustrating a method for providing recorded inner-body sounds that may be used during a face-to-face diagnosis of an asthmatic patient according to various embodiments of the invention.

### DETAILED DESCRIPTION OF THE PREFERRED EMBODIMENTS

A system, apparatus and method are described for recording, processing, measuring and transmitting electrical data relating to audible signals originating from within a human body. Acoustic signals originating from a human body are detected by a hand-held device that converts these acoustic signals into corresponding electrical signals. Within the housing of this hand-held device, these signals are processed in the electrical domain, such as filtering, amplifying, storing and analyzing, so that they may be provided in a preferred output format. In certain embodiments of the invention, a speaker on the device housing outputs audible signals relating to the detected inner-body acoustic signals. In other embodiments, a port is provided that outputs electrical signals which may be transmitted over a connection to a distant remote device or stored on a computing device external to the hand-held device. In yet other embodiments, an antenna is provided that transmits a wireless signal relating to the detected inner-body acoustic signals. These various embodiments of the invention allow for a more effective diagnosis of medical conditions, such as asthma or heart ailments, by a remote medical professional or recording of inner-body acoustic signals which may be later analyzed by the medical professional.

In the following description, for purpose of explanation, specific details are set forth in order to provide an understanding of the invention. It will be apparent, however, to one skilled in the art that the invention may be practiced without these details. One skilled in the art will recognize that embodiments of the present invention, some of which are described below, may be incorporated into a number of different electrical components, circuits, devices and systems. Structures and devices shown below in block diagram are illustrative of exemplary embodiments of the invention and are meant to avoid obscuring the invention. Furthermore, connections between components within the figures are not intended to be limited to direct connections. Rather, connections between these components may be modified, re-formatted or otherwise changed by intermediary components.

Reference in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment.

A. Overview

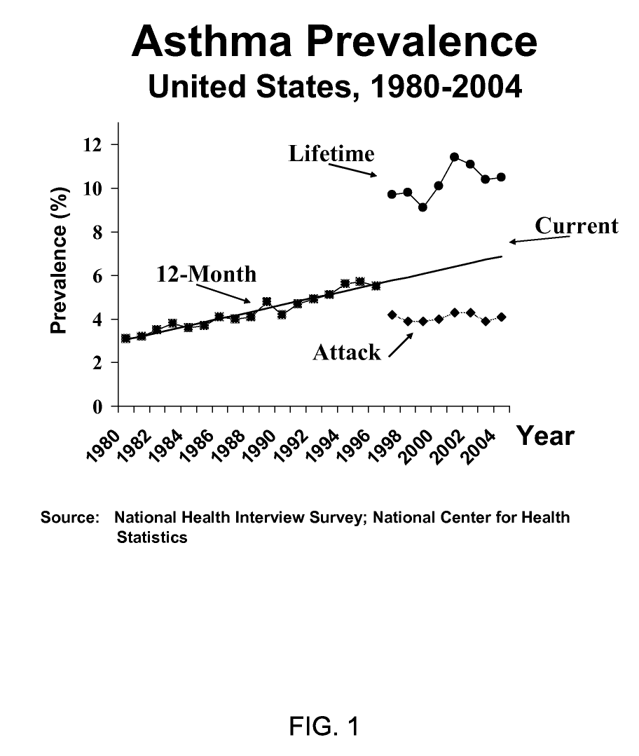

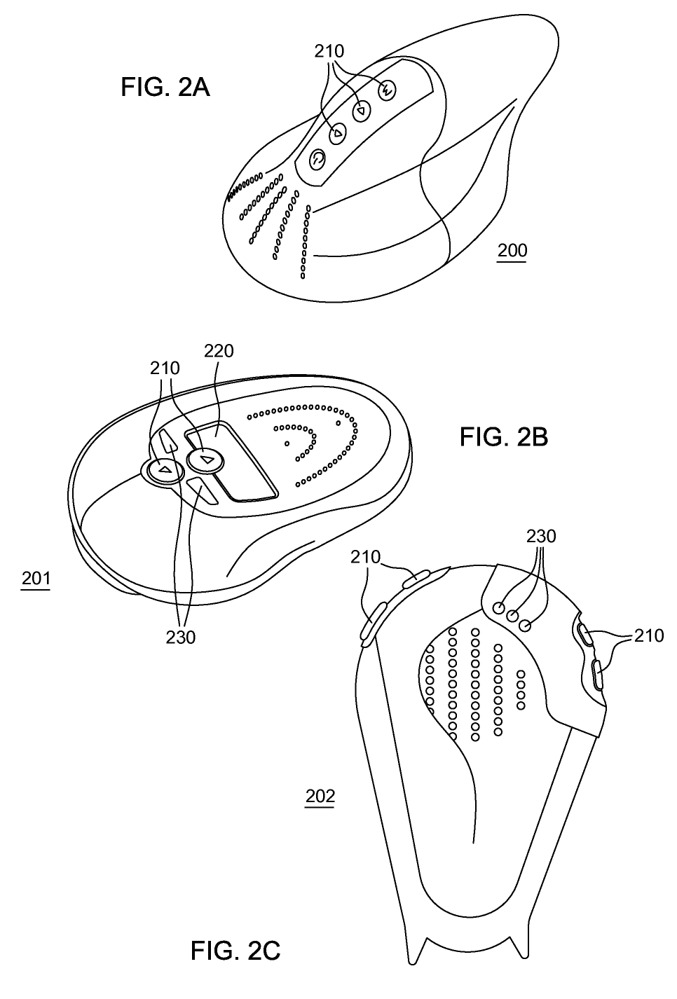

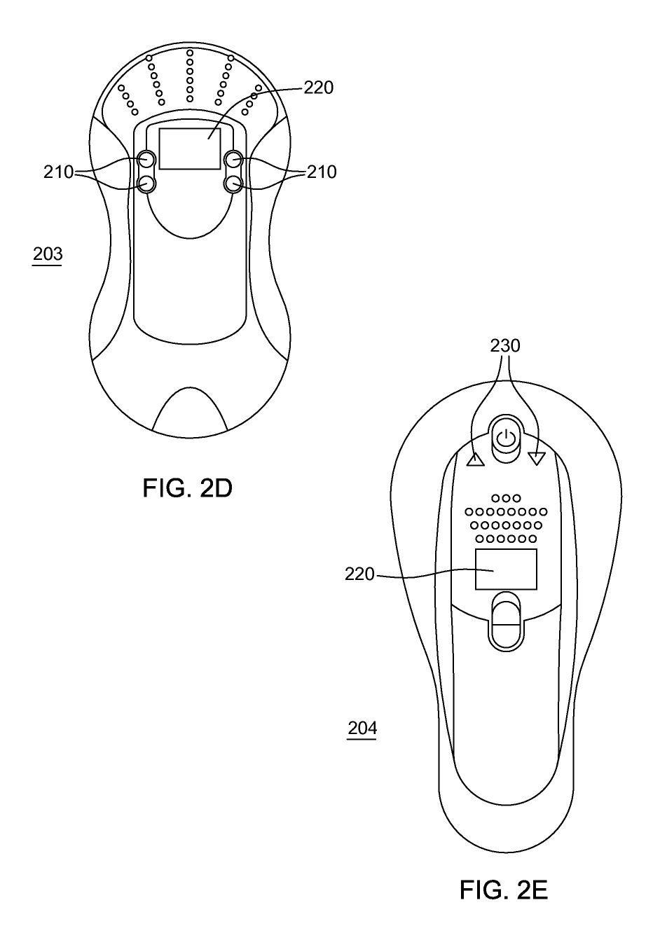

FIGS. 2A-2E illustrate design examples of an inner-body sound monitor and recording apparatus in accordance with various embodiments of the invention. The apparatuses are designed to allow a non-medical professional to use an apparatus to monitor acoustic noise within a human body. For example, an apparatus may be used to receive, amplify and audibly output breathing sounds of an asthmatic child. Other functionality, such as inner-body sound recording and transmission of this sound to a remote device, may also be provided within various designs of the apparatus.

The apparatus may also be designed to minimize the cost of manufacturing and selling the apparatus. In various embodiments, all of the functional components are contained within a single housing that may be easily held against a skin surface. The housing may be designed to allow it to be grasped by a single hand and pressed against the skin surface. The housing may also contain buttons, knobs or other command components that allow an individual to perform certain operations or control the volume of a speaker playback. The housing may also contain user interfaces, such as an LCD or LED screen, that provide information to the user.

Referring to FIGS. 2A, 2B, 2C, 2D and 2E, an inner-body sound monitor and recording apparatus 200-204 may have various functional designs and features, and does not have a “tail” like a stethoscope. The apparatus 200-204 may have various user interface components that allow a user to provide data to or receive data from the apparatus 200-204. For example, certain buttons 210 on the apparatus may provide for volume control, allow a user to input a date and/or time stamp, allow a user to input a location on the body from which a recording was received, and other data that may be relevant to a particular recording. A screen 220 may also be provided to communicate information to a user and/or an interface for a user to input data. The screen 220 may be an LCD display (and may or may not be touch sensitive) or a LED display. The apparatus 200-204 may also have a number of different signatory lights 230 to identify a user certain information. For example, lights may be used to signal a user when the apparatus 200-204 is ready to record or measure sound, and signal when a recording has completed.

In other embodiments, a larger LCD screen, such as one typically used within a personal digital assistant (“PDA”), may be integrated within the apparatus 200-204. Functionality may be implemented within the various different embodiments of the invention, examples of which are provided below.

Referring to FIG. 2A, the apparatus 200 is designed to allow a user to easily grip the housing. A top small part is used to fit between a user's thrum and index finger, and is small enough for a small hand to grasp it while still allowing larger hands to use it. The bottom of the housing is designed to locate or pinpoint a location of diagnosis on a patient. The patient's index finger is positioned on the middle of the apparatus 200, which has a convenient one button control. The apparatus 200 is also designed to be symmetrical which would allow use by either a left hand or a right hand.

In other embodiments of the invention, the apparatus 201-203 may have a screen on top that provides information to a user and may allow a user to input information. For example, the screen may be a touch screen or a virtual keypad. The top right has two button for various control functionality and has a side view which visually shows a smaller skin contact to aid in locating or pinpointing a listening/recording location on a body. Referring to apparatus 203, the housing is shaped like a computer mouse for comfortable use. The housing may include a wheel, similar to a mouse, which may provide certain functions. Other design features, such as right and left click buttons may be implemented within the apparatus 203 to make using the device more familiar to a user.

In certain embodiments, the center of the apparatus 200-204 may be etched so that a user's index finger can press the apparatus 200-204 firmly against the skin.

The apparatus is used by pressing an input surface against a skin surface of a patient. The housing surface that is pressed against the skin may be flat or contoured depending on various embodiments of the invention. Depending on what acoustic sounds are to be monitored, the input surface of the apparatus should be placed against an appropriate skin region of the body. For example, if an asthma attack is to be monitored and recorded, the apparatus should be pressed against certain locations on the patient's chest and/or back so that the desired acoustic noise is recorded within the apparatus. Sounds from the lung area are accurately recorded and/or transmitted to a remote location for analysis by a trained medical professional. Based on the playback of the sounds, a doctor can base a diagnosis on the recorded sounds instead of verbal descriptions of the sounds by the patient, parent or other untrained individual.

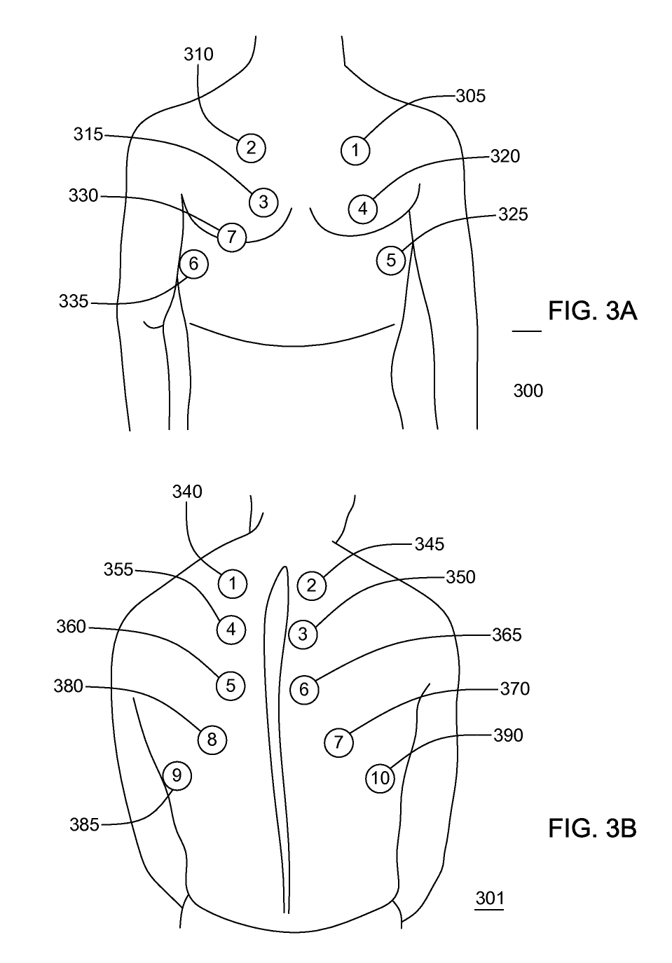

FIG. 3A shows certain locations on a patient's chest where the apparatus input surface may be pressed against to record different breathing sounds that are relevant to an asthma attack diagnosis. The apparatus input surface is pressed against the bare skin at one or more of these locations for a minimum amount of time. Various embodiments of the apparatus may have an input so that an individual can associate a particular location on the body with a recording stored within the apparatus.

In one embodiment of the invention, a first set of recordings are taken at a first location 305 and a second location 310 at the apices of the lung just below the collar bones. Third and fourth recordings 315, 320 are taken at locations below the first and second locations. Additional recordings 325, 330, 335 may be taken on either side of the ribs, parallel to the bottom of the sternum or mid-way from the base of the neck and the belly button. The sequence of recordings may be tailored to a particular patient or may be generalized across multiple patients. This sequence of recordings and/or measurements may be given to a medical professional in order to provide detailed information of breathing symptoms of a patient.

FIG. 3B illustrates locations on a back that may also provide monitoring points relevant to asthma diagnoses. In a similar manner as the chest, a plurality of recordings may be taken on the back. The sequence of these recordings may be designed specifically for a patient or generalized across multiple patients. There may be certain advantages to taking the recordings on a patient's back but would obviously require a care giver or third party to physically take the recordings.

One skilled in the art will recognize that various locations and sequences of measurements may be performed in order to obtain relevant information different diagnoses. Although the above-description relates to asthma diagnosis and treatment, numerous different health issues, for which inner-body sounds may be relevant, may be addressed by various embodiments of the invention.

B. Inner-Body Sound Monitor and Recording Device

An inner-body sound monitor and recording apparatus may be an analog or digital device. The apparatus is designed to allow a non-medical professional to record sound originating within a human body and obtain other associated medical information for later use by a medical professional.

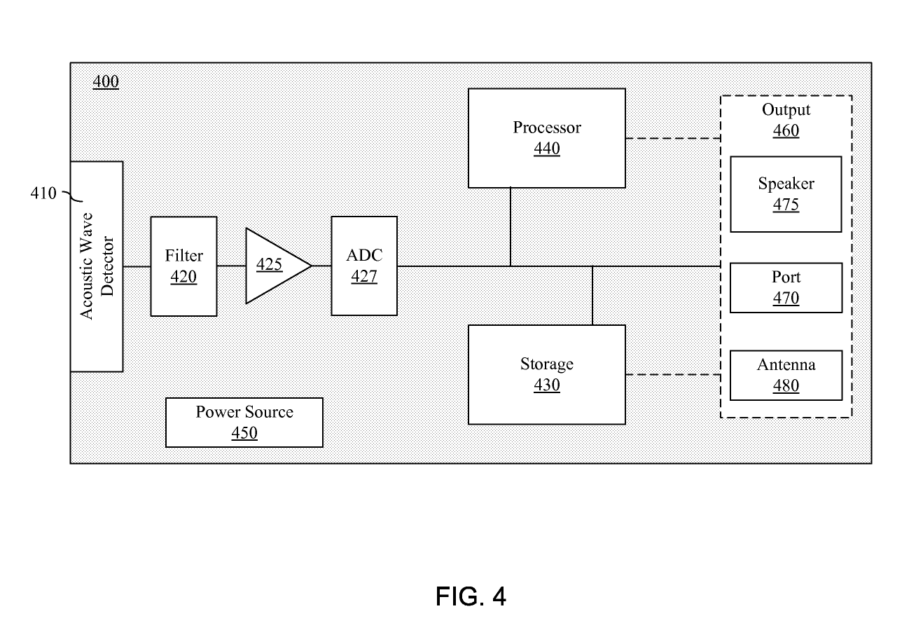

FIG. 4 illustrates a digital inner-body sound monitor and recording apparatus 400 according to various embodiments of the invention. The apparatus comprises an acoustic wave detector 410 located on an input surface of the apparatus 400. The detector 410 may be a microphone, transducer or other component that receives an acoustic signal and coverts it into an analog electrical signal. The input surface may be flat or curved to enable the detector to be pressed against a skin surface so that noise within a human body may be properly detected. A second input may be located on the apparatus 400 that allows a user to provide information. This second input may include buttons, a touch sensitive LCD screen, keypad, or other input device that allows a user to provide information that may be relevant to a recording of inner-body sounds.

A filter 420 is coupled to the detector 410 and removes undesirable noise on the electrical signal. In various embodiments, the filter 420 may be a bandpass or lowpass filter. For example, the filter 420 may be a lowpass filter that passes frequencies below approximately 200 Hz. An amplifier 425 is coupled to the filter 420 and provides a gain on the electrical signal that is output from the filter 420. The gain across the amplifier 425 may be fixed or adjusted according to the type of noise being detected and the environment in which the detection is occurring. An analog-to-digital converter (“ADC”) 427 is coupled to receive the amplified analog signal and converts it to a digital signal for subsequent processing. The converter 427 characteristics, such as sampling rate and thresholds, may be adjusted depending on numerous factors including the anticipated signal-to-noise ratio of the analog signal.

This digital signal may be communicated to subsequent components by a bus or digital connections. This digital information may be provided to a storage device 430, located within the device housing, that records this information so that it may be subsequently provided to a medical professional. This storage 430 may be volatile or non-volatile memory and include various types such as flash, RAM, ROM, EEPROM and other digital storage mediums. Additionally, the digital information may be compressed prior to storage using known compression techniques.

A processor 440 is also coupled to receive this digital information either from a bus, directly from the converter 427, or from the storage device 430. The processor 440 may also control the elements within the apparatus 400. The processor 440 may command storage of other information associated with the detected noise, such as the time of detection and/or the body location at which the noise was detected. The processing functions and operations may be realized in hardware, software or firmware and be implemented in such devices as FPGAs, CPUs, DSPs, and other processing devices known within the art. Additionally, as will be discussed later, the processor 440 may perform various analysis operations on the digital information to provide additional information to a user about the detected acoustic signals.

An output 460 is coupled to receive the stored digital information, including the digitized inner-body sounds, and provide it to a user. The connection may be a bus, a direct connection to the storage device 430, or a direct connection to the processor 440. Various types of outputs may be used depending on the particular embodiment that is realized within the apparatus 400. For example, a speaker 475 on the apparatus housing may provide an audible playback representation of the detected inner-body noise. In other embodiments of the invention, a transmission port or ports 470 may be provided so that an electrical output signal may be transmitted from the apparatus 400. These ports 470 may include a USB port, firewire port, Ethernet port, cable port, telephone line port, etc. Additionally, the ports 470 may include a receptacle for headphones that would allow a medical professional to listen to detected inner-body noise in real time. In yet other embodiments, an antenna 480 may be used as an output that transmits a wireless output signal. For example, a headset my wirelessly connect with the apparatus using a wireless transmission technology or protocol such as Bluetooth. One skilled in the art will recognize that other types of output types may be incorporated within the output 460.

The apparatus 400 may also provide a user the ability to input information such as a time stamp, observations, body locations where a recording was taken, questions, or any other relevant information. These inputs may be realizes as buttons, an LCD screen, another microphone, a keypad or any other type of data entry component.

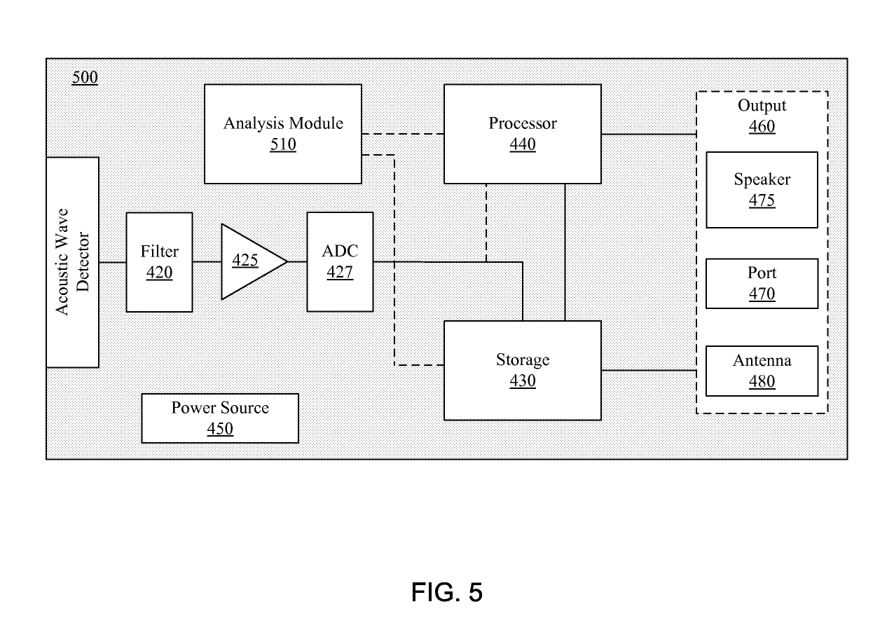

FIG. 5 illustrates a digital inner-body sound monitor and recording apparatus 500, including dynamic analysis of detected inner-body noise functionality, according to various embodiments of the invention. An analysis module 510 provides analysis functionality to the apparatus to assist a medical professional in a diagnosis or provide more information to a non-medical professional. Depending on the implementation of the apparatus 500, the analysis module 510 may be communicatively coupled to the processor 440, the storage 430 or both. In various embodiments of the invention, the analysis functionality may thus be performed independently by the analysis module 510, or collectively by the processor 440 and analysis module 510. In other embodiments, the analysis module 510 may be integrated within the processor 440.

The analysis module 510 may provide characterizations of stored inner-body sound based on an analysis of the recorded sound. In one embodiment, stored inner-body sound is compared to a plurality of different patterns or diagnoses footprints to determine if the stored sound is indicative of one or more particular symptoms. This analysis may include a frequency pattern of the recorded sound, an intensity pattern of the recorded sound, and other pattern characteristics that may indicate a particular symptom. These patterns or footprints may be identified overtime by storing and analyzing digitized patient inner-body sounds.

In the case of asthma related noise, digitized inner-body noise is compared to different patterns to determine whether the noise is wheezing, crackling or other asthmatic symptoms. These patterns may be generated based on provided by an external source into the apparatus. In certain embodiments, the patterns may be dynamically updated by recording asthmatic sounds from a patient and then classifying those particular sounds within the device. As a result, patient specific digital footprints are stored within a patient's apparatus 500.

The analysis module 510 may also provide an analysis of information provided by a user through a user interface on the apparatus 500. This information may include telephone numbers to call, instructions for initial treatments, and other instructions that may be relevant to a non-medical professional that is assisting a patient. The analysis module 510 may also access historical readings to identify when inner-body noise would be considered abnormal based on a patient's medical history.

In the case of asthma patients, the analysis module 510 may also provide a ranking or scale of the severity of an attack. In one embodiment, the analysis module 510 provides a number assessment, between 1 and 10, describing the severity of the asthma attack. This number may be generated based on the characteristics of the recorded asthmatic noises such as intensity, frequency footprint, etc. One skilled will recognize that other analyses may be applied and realized within the analysis module 510; all of which are intended to fall within the scope of the present invention.

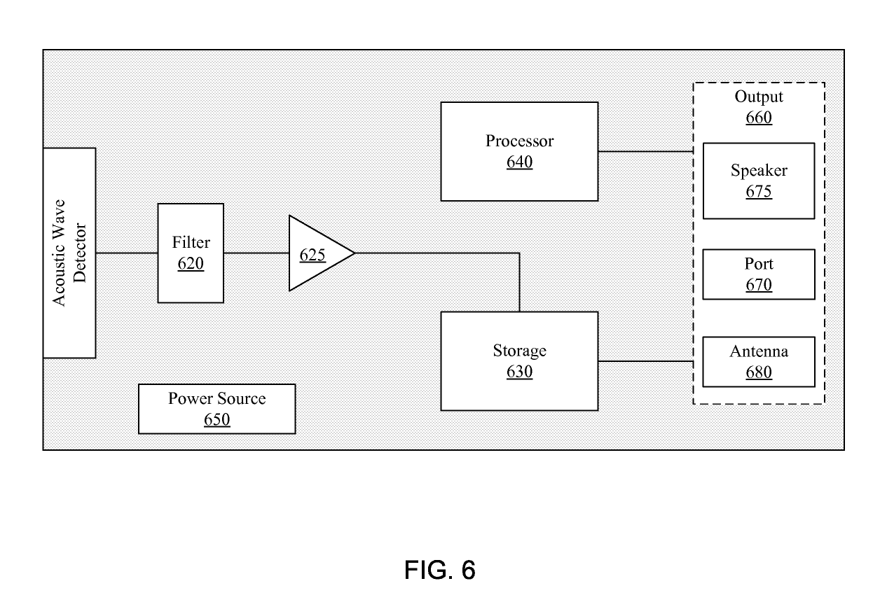

FIG. 6 illustrates an analog inner-body sound monitor and recording apparatus 600 according to various embodiments of the invention. The apparatus 600 comprises an acoustic wave detector 610 located on an input surface of the apparatus 600. The detector 610 may be a microphone, transducer or other component that receives an acoustic signal and coverts it into an analog electrical signal. Similar to the digital apparatus 400, the input surface may be flat or curved to enable the detector 610 to be pressed against a skin surface so that noise within a human body may be properly detected.

An analog filter 620 is coupled to the detector 610 and removes undesirable noise on the electrical signal. In various embodiments, the filter 620 may be a bandpass or lowpass filter. For example, the filter 620 may be a lowpass filter that passes frequencies below approximately 200 Hz. An amplifier 625 is coupled to the filter 620 and provides a gain on the electrical signal that is output from the filter 620. The gain across the amplifier 625 may be fixed or adjusted according to the type of noise being detected and the environment in which the detection is occurring.

An analog storage device 630 is coupled to the amplifier 625 so that the detected noise may be stored internally within the apparatus 600. The analog storage device 630 may be magnetic tapes and is coupled to an analog processing device or a state machine 640. The processor or state machine 640 and the storage device 630 are coupled to an output 660 that allows a user to hear or store the recorded inner-body noise. This output 660 may comprise a speaker 675, a port 670, an antenna 680 or other output component. In one embodiment of the invention, a speaker 675 is provided that produces an audible reproduction of the stored inner-body noise. In other embodiments, a port 670 is provided that allows the stored inner-body to be transmitted as an electrical signal. This port 670 may be a jack for headphones, an Ethernet or other networking port, a cable port, a USB port, a firewire port, or any other port known within the art. In yet other embodiments, the output 660 may be an antenna 680 that allows the stored inner-body noise to be transmitted wirelessly to a remote location.

C. Remote Analysis of Inner-Body Sound

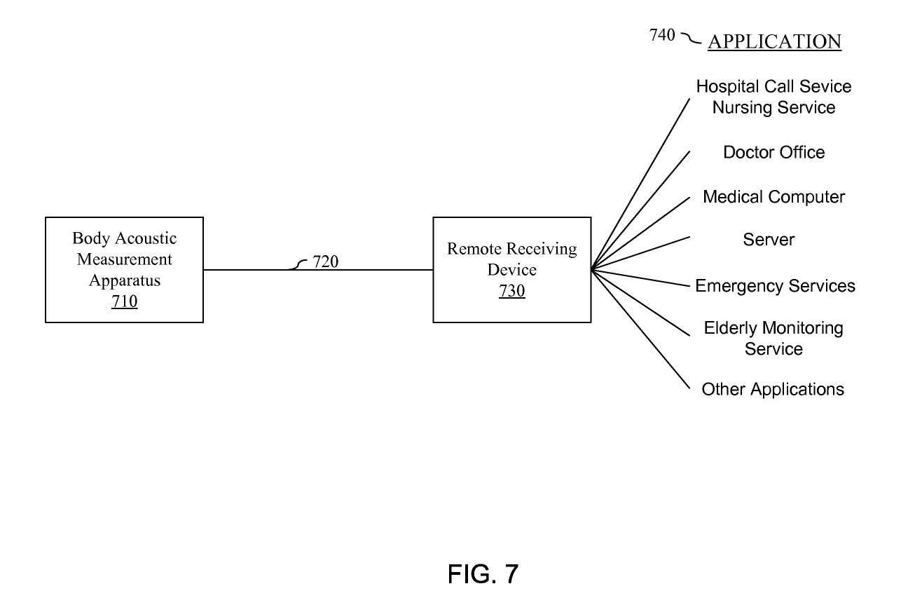

FIG. 7 illustrates a system for remotely receiving recorded inner-body sounds according to various embodiments of the invention. An inner-body sound monitor and recording apparatus 710 is coupled via transmission connection 720 to a remote receiving device 730. Various applications, including those listed as APPLICATIONS 740, may be applied to the system.

As previously mentioned, the inner-body sound monitor and recording apparatus 710 has an output, such as a port or antenna, that allows it to communicate with a remote receiving device 730. This communication may occur on connections such as networks, wireless links, phone connections, cable connections, or other communication media.

The remote receiving device 730 may be a server, a computer, a telephone, a personal digital assistant, or other electronic device that can receive data from the inner-body sound monitor and recording apparatus 710. The remote receive device 730 may store this data, display this data, generate an audible signal from this data, analyze this data, or otherwise process the data.

The remote receiving device 730 may support numerous applications that used this transmitted data, of which FIG. 7 provides a small subset 740 of these applications. Inner-body sound data may be remotely used by a hospital call service or nursing service to provide more accurate information about a patient's condition so that a well-informed decision may be made. For example, a call center nurse may remotely listen to a child's asthma attack to determine whether the child should go to an emergency room.

Inner-body sound may also be used and/or monitored remotely by a doctor's office. In one example, a patient may provide a doctor's office a recording of inner-body noise such as heart sounds or breathing sounds, to allow the office to consistently check on a particular symptom of the patient. These inner-body noises may also be stored and catalogued to create a medical history of a particular symptom or symptoms of a patient. In addition, a medical professional may receive this data and/or listen to the inner-body sounds when he/she is away from the office. As such, the medical professional would be able to remotely diagnosis a patient while away from the medical office.

The remote receiving device 730 may also support medical analysis, storage and other processes that aid in the treatment of a patient. For example, the analyses described-above in relation to the analysis module 510 may be integrated with the remote receiving device 730 to allow identification, classification and other analyses of inner-body sounds. As a result, a centrally located computing device could receive data from multiple patients, analyze the data and provide an output to a medical professional and/or maintain historical data on each of the patients.

The remote receiving device 730 may interface with a server for various purposes including storage, automated analysis and providing remote access to the data by a user. For example, a doctor may be able to remotely access the server and retrieve or analyze the stored data therein. The functions described in relation to the medical computer may also be provided in the server.

The remote receiving device 730 may also support emergency services in various capacities. For example, the remote receiving device 730 could allow a 911 operator to remotely hear certain inner-body sounds. In addition, historical recordings may also be accessed by a 911 operator or emergency medical technician to provide additional information for a diagnosis or treatment of a patient. The receiving device 730 may also be used to receive inner-body noise from an emergency medical technician in transit with a patient and these transmitted noises to a doctor. One skilled in the art will recognize that other emergency services may be supported by various embodiments of the present invention.

The remote receiving device 730 may also be used to assist elderly monitoring services. In one embodiment, the inner-body sound monitor and recording apparatus 710 is used by an elderly patient to provide his/her doctor health information. For example, an elderly patient may use the apparatus 710 to provide information to a doctor in the event of an emergency. In another example, a schedule may be designed for an elderly patient to take a recording and provide it to a medical professional and/or medical computer. As a result, a historical record may be preserved for an elderly patient.

The remote receiving device 730 may also support a browser-based user interface that allows localized access and information collection and distribution of data stored within the apparatus 710. A corresponding server may support this browser-based user interface and allow medical professionals, patients, and other individuals to access, store and otherwise process information on the server. In addition, various rights may be associated with a user so that the information may be protected and distribution of the information tailored to particular individuals.

The above-described applications are intended to be exemplary and not limiting. One skilled in the art will recognize that other applications may be supported by the inner-body sound monitor and recording apparatus 710 and the remote receiving device 730.

D. Methods of Monitoring and Storing Inner-Body Sounds

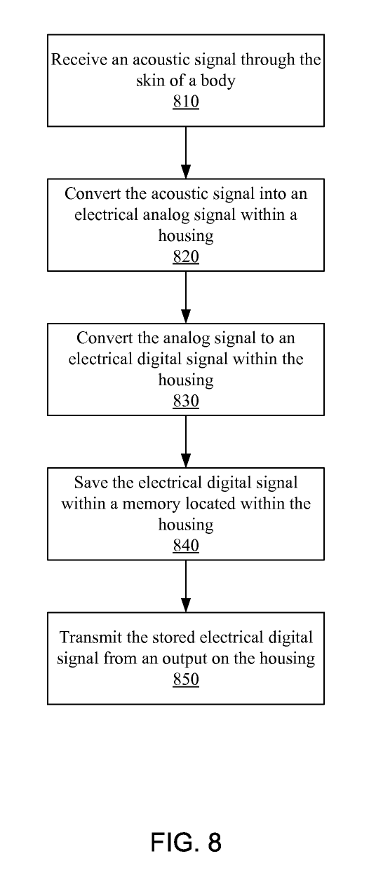

FIG. 8 illustrates a general method, independent of structure, for allowing a non-medical individual to store and transmit inner-body sounds to a remote device according to various embodiments of the invention. A single-housing device design allows a non-medical individual to record inner-body sounds. Output functionality is provided that allows the stored data to be retrieved such as through an audible signal from a speaker, an electrical signal from a port, or a wireless signal from an antennal.

An acoustic signal or sound is received 810 by a hand-held, single-housing device that allows a non-medical user to comfortably use it. The acoustic signal is converted 820, within the single-housing device, into an electrical analog signal so that it may be processed within the electrical domain.

The analog electrical signal is converted 830, also within the single-housing device, into an electrical digital signal. Thereafter, the digital signal is stored 840 within a memory device located in the single-housing device. In one embodiment of the invention, the stored information is electronically transmitted 850 from a port on the single housing device to a remote device so that a medical professional may analyze, store or otherwise process the data related to the inner-body sounds.

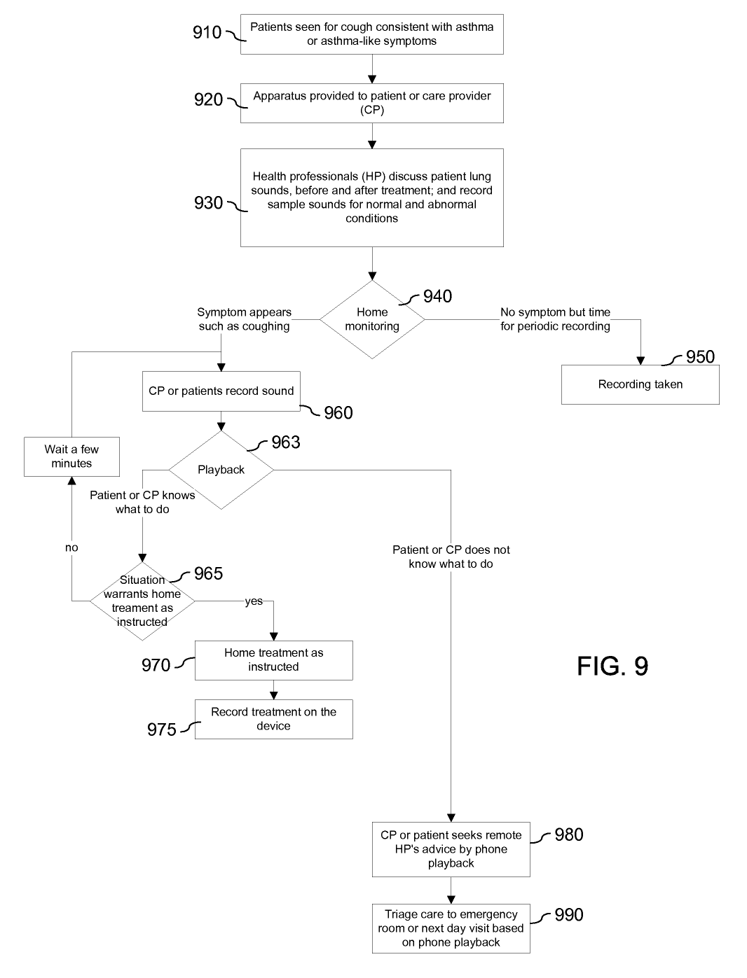

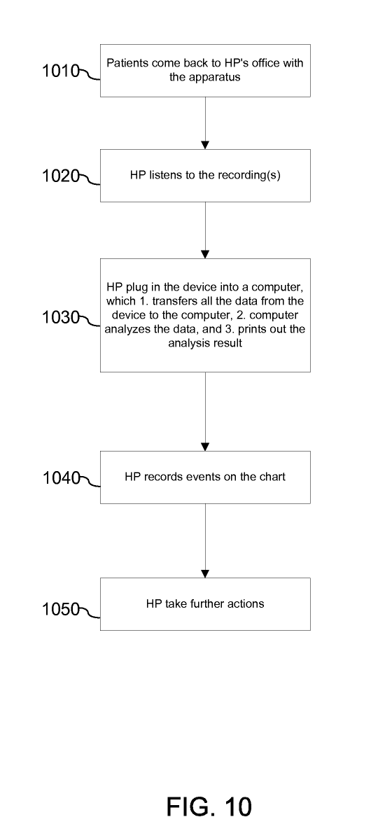

FIGS. 9 and 10 illustrate a more specific method, independent of structure, for treating an asthmatic patient in which a portion of the treatment includes a non-medical individual recording and storing breathing/lung sounds of a patient according to various embodiments of the invention.

According to various embodiments of the invention, a patient that is seen by a medical profession 910 for a cough or other asthma-like symptoms. The medical profession provides 920 the patient with an inner-body sound monitor and recording apparatus. The apparatus may be pre-configured with standard asthmatic information and configurations, or may be configured to a specific patient. For example, the apparatus may be configured with generalized comparable asthmatic sounds or may contain the patient's historical sounds as well as a doctor's characterization of the sounds. The patient is given instructions on how to use the apparatus 930 by the medical professional or support staff.

A patient is able to use the apparatus at home 940 and take consistent measurements or recordings 950 according to a schedule or on a consistent regular basis. These recordings may or may not occur during an asthma attack.

The patient is also able to take measurements or recordings 960 when an asthmatic symptom appears, such as an asthma attack. The apparatus allows a patient or care giver to record inner-body sounds emanating from the patient's lungs and subsequently listen to them. In the case of asthma, the patient is able to listen 963 to these sounds, such as wheezing, to determine whether home treatment is appropriate 965. Based on an analysis of the sounds, the patient may determine an appropriate course of home treatment, act accordingly 970 (such as using an inhaler), and record the treatment information on the apparatus 975.

If after listening to the recorded inner-body sound, a patient is unsure what the next step should be, he/she may call 980 a medical professional, such as a nurse or doctor. The patient may describe his/her condition over the phone and provide the recordings to the medical professional to help in the diagnosis. These recordings may be provided to the medical professional using various methods such as transmitting the recording in an electrical signal to the medical professional or providing an audible signal (such as holding the apparatus next to the phone and replaying the recording).

The recorded inner-body sounds assist the medical professional in accurately diagnosing the patient's condition and determining an appropriate course of action. For example, triage care or next-day visit may be scheduled 990 in response to listening to the recorded sounds. As a result, patients are more efficiently and accurately diagnosed using the recorded inner-body sounds. This improvement in efficiency and accuracy reduces the cost of caring for asthmatic patients as well as improves the quality of care provided to them.

The apparatus may also be used to supplement information provided to a doctor when an asthmatic patient returns to a doctor's office. For example, when a patient returns 1010, the doctor may listen 1020 to recorded inner-body sounds, including those that were taken during an asthma attack and those taken in accordance with a schedule, and/or further process the recordings. For example, the doctor may plug 1030 the apparatus into a computing device and transfer data, such as recorded breathing symptoms, into the computing device. The computing device may have analysis functionality that analyzes the recorded inner-body sounds and provides the medical professional an output, such as an historical accounting as well as abnormalities within the data. For example, an analysis may include identifying abnormal sounds relative to a differential from baseline sounds. As discussed above, the analysis may also include temporal or other associated recordable changes within sounds. Advanced analysis may also be provided which includes indicators for severity based on and body mass index.

The recorded inner-body sounds, and any associated data therewith, may be stored 1040 within the computing device. As a result, a medical record of the patient may be supplemented with these historical inner-body sound recordings, and any analysis thereof, to provide a more complete description of asthmatic symptoms of a patient. At least partially based on these recordings, a medical professional can provide 1050 a more accurate diagnosis and treatment of the patient's asthma.

The foregoing description of the invention has been described for purposes of clarity and understanding. It is not intended to limit the invention to the precise form disclosed. Various modifications may be possible within the scope and equivalence of the appended claims.

We claim:

1. An inner-body sound monitor and recording apparatus comprising:
a housing having an input surface that is placed directly on a skin surface;
an acoustic signal detector, located on the input surface, that receives an acoustic signal from the skin surface and generates an analog electrical signal;
an analog-to-digital converter, coupled to the acoustic signal detector and within the housing, that converts the analog electrical signal to an digital signal;
a storage device, coupled to the analog-to-digital converter and within the housing, that stores the digital signal;
an output, coupled to the storage device, that provides an output signal related to the acoustic signal; and
a processor coupled to the analog-to-digital converter, the storage device, the output and within the housing, that controls the storage of the digital signal and the generation of the output signal.

2. The apparatus of claim 1 further comprising a filter, coupled between the acoustic signal detector and the analog-to-digital converter, that removes at least one frequency from the analog electrical signal.

3. The apparatus of claim 2 wherein the filter is a lowpass filter.

4. The apparatus of claim 2 further comprising an amplifier, coupled between the filter and the analog-to-digital converter, that applies a gain to the analog electrical signal.

5. The apparatus of claim 1 wherein the output comprises a speaker that generates an audible signal from data stored within the storage device.

6. The apparatus of claim 1 wherein the output comprises an antenna that generates a wireless signal from data stored within the storage device.

7. The apparatus of claim 6 wherein the wireless signal is a Bluetooth signal.

8. The apparatus of claim 1 wherein the output comprises a port that transmits an electrical output signal from data stored within the storage device.

9. The apparatus of claim 8 wherein the port is an Ethernet port that transmits the electrical output signal onto a network.

10. The apparatus of claim 8 wherein the port is a headphone jack that transmits an electrical signal to a headphone.

11. The apparatus of claim 8 wherein the port is a telephone jack that transmits an electrical signal to a telephone device.

12. The apparatus of claim 1 further comprising an analysis module, coupled to the storage device, that analyzes data stored within the storage device and provides an output to a user.

13. The apparatus of claim 12 wherein the analysis module compares the data stored within the storage device to a plurality of digital patterns in order to characterize the acoustic signal associated with the stored data.

14. The apparatus of claim 12 wherein the analysis module compares the data stored within the storage device to at least one threshold to determine an intensity of the acoustics signal associated with the stored data.

15. A system for remotely monitoring a patient, the system comprising:
an inner-body sound monitor and recording apparatus, having a single housing and output port, that records inner-body sounds from a patient and transmits an electrical signal, derived from the recorded inner-body sounds, onto a communications line; and
a remote receiving device, coupled to the communications line, that receives the electrical signal.

16. The system of claim 15 wherein the remote receiving device is a computing device that stores and analyzes the electrical signal in order to build a patient history of recorded inner-body sounds.

17. The system of claim 15 wherein the remote receiving device is a computing device on which a medical professional may listen to recorded inner-body sounds of a patient during a diagnosis of the patient's condition.

18. The system of claim 15 wherein the remote receiving device is a computing device on which an emergency call operator may listen to recorded inner-body sounds of a patient during an emergency telephone call.

19. A method for recording inner-body sounds of a patient, the method comprising:
detecting an acoustic signal originating from inside a body by positioning a single-housing apparatus on a skin surface;
converting the acoustic signal to an electrical signal;
storing the electrical signal within the single-housing apparatus;
providing a time stamp associated with the stored electrical signal; and
outputting a signal to enable a playback of the acoustic signal from inside the body.

20. The method of claim 19 wherein the outputted signal is an electrical signal that is transmitted to a remote location for storage.
